(19) United States
(12) Patent Application Publication
(10) Pub. No.: US 2011/0046217 A1
(43) Pub. Date: Feb. 24, 2011

(54) USE OF 2,2'-CYCLOLIGNANS FOR INDUCING, RESTORING OR STIMULATING THE PIGMENTATION OF THE SKIN, HAIR OR HAIRS

(76) Inventors: Philippe BERNARD, La Ferte Saint Aubin (FR); Laurent RIOS, Azuat La Combelle (FR); Jean-Yves BERTHON, Romagnat (FR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/674,684

(22) PCT Filed: Aug. 22, 2008
(86) PCT No.: PCT/EP2008/061004

§ 371 Date: Apr. 6, 2010

(30) Foreign Application Priority Data
Aug. 24, 2007 (FR) 07/06004

(51) Int. Cl. A61K 8/49; A61K 31/36; A61P 17/00; A61Q 19/00

(52) U.S. Cl. 514/463

(57) ABSTRACT

The invention relates to the use of 2,2′-cyclolignanes in the cosmetic or pharmaceutical field for inducing, restoring or stimulating the pigmentation of the skin, hair or hairs.

The present invention relates to the use of 2,2′-cyclolignans for the preparation of a composition intended for inducing, restoring or stimulating the pigmentation of the skin, body hair or head hair.

The color of human skin depends on many factors and especially on race and sex, but also on environmental factors (season, exposure to sunlight); it is mainly dependent on the nature and concentration of melanin produced by the melanocytes. Melanocytes are specialized cells that synthesize melanin, using particular organelles, the melanosomes. Certain individuals naturally or accidentally have more or less localized pigmentation defects, requiring palliative local treatments, or demand more general treatments, for stimulating natural pigmentation.

Pigmentation is a natural effective protection against the harmful effects of ultraviolet radiation and against photoaging of the skin in general. Skin pigmentation is also a protection against the onset of skin cancers; for the same magnitude of exposure to sunlight, dark-skinned individuals and ethnic groups develop far fewer skin cancers than individuals with pale skin.

Similarly, the color of body hair and head hair is due to melanin. At various periods in their life, especially during aging, some individuals develop gradual depigmentation of their head hair, with a reduction in or even the stoppage of the processes of melanogenesis in the melanocytes associated with the hair bulb. It would be very advantageous to be able to propose preventative or curative treatments capable of maintaining the process of pigmentation of the hair or of stimulating melanogenesis and pigmentation of hair with a tendency towards graying.

Exposure to sunlight and UV radiation have harmful effects on the hair, not only on the hair stem (oxidation and bleaching), but also, and more destructively, on the follicle bulb, which may lead to loss of the hair. The recovery or stimulation of hair follicle pigmentation is capable of limiting the loss of the hair or of stimulating its regrowth.

The mechanism of formation of skin pigmentation is complex and schematically involves the following main steps:

tyrosine→dopa→dopaquinone→dopachrome→melanin. Melanin is stored in organites or melanosomes, and then transferred to the neighboring keratinocytes.

Each of these steps is essential to pigmentation. Tyrosinase (monophenol dihydroxyl phenylalanine: oxygen oxidoreductase EC 1.14.18.1) is the first enzyme involved in this sequence of reactions. It especially catalyzes the reaction for transformation of tyrosine to dopa (dihydroxyphenylalanine) by virtue of its hydroxylase activity, and the reaction for transformation of dopa to dopaquinone via its oxidase activity. This tyrosinase acts only when it is in the mature state, under the action of certain biological factors; signaling via specific receptors such as the melanocortin receptors (MCR) is involved for induction of the melanin synthesis process by the melanocytes, especially the receptor MC1R.

In the epidermis, the melanocyte is involved in the epidermal melanic unit, which comprises a melanocyte surrounded by about 36 neighboring keratinocytes. All individuals, without distinction as to phototype, have approximately the same number of melanocytes for a given area of skin. The ethnic differences, in terms of pigmentation, are not due to the number of melanocytes, but to the properties of their melanosomes. The melanosomes are aggregated as complexes and are of small size. They are highly specialized organelles whose sole function is to produce melanin. Gradually, as melanin is synthesized in the melanosomes, they move from the perinuclear region to the extremity of the melanocytes' dendrites. Via phagocytosis, the extremity of the dendrites is captured by the keratinocytes, and the melanosomes are redistributed in the keratinocytes. The dendritic extensions of the melanocytes, and the phagocytic activity of the keratinocytes, thus play an essential role in the transfer of melanin. Melanosome transfer is a phagocytic phenomenon considered as standard, which involves receptors known as the “protease-activated receptor 2” (PAR-2).

Although the level of melanin varies from one population to another, the amount of tyrosinase does not vary significantly and the level of tyrosinase messenger RNA is identical in white or black skin. The variations in melanogenesis are thus due to variations either in tyrosinase activity or in the capacity of the keratinocytes to phagocytose the melanosomes. This indicates that the keratinocyte is a major player in pigmentation; 1) it is quantitatively the major representative of the melanic unit, and is also the agent that influences, via information molecules (cytokines and hormones), a large proportion of the melanogenic activity; 2) it is its capacity for phagocytosis, combined with an adequate presentation of the melanosomes, in a dense dendritic network, which allows optimum distribution of melanin in the epidermis and pigmentation. A substance is recognized as being pro-pigmenting if it acts directly or indirectly on activation of the melanin synthesis process, and/or if it stimulates the melanosome phagocytosis capacity by the keratinocytes.

Substances such as α-melanotropin (α-melanocyte-stimulating hormone, α-MSH) and corticotropin (adrenocorticotropic hormone, ACTH) stimulate melanin proliferation and synthesis by the melanocytes, via binding to specific receptors, especially the receptor MC1-R. However, few natural inducers are currently available and used for natural melanic pigmentation of the skin or the hair.

International patent application WO 2005/044289 describes the use of an extract of the fruit of Schisandra chinesis for cosmetic purposes for inhibiting the synthesis of melanin and thus whitening the skin. Similarly, international patent application WO 01/41778 discloses cosmetic compositions intended for inhibiting the synthesis of melanin and which contain gomisin N or g-schizandrin or else an extract of Schisandra. This document claims the use of such cosmetic compositions, especially for whitening the skin.

Japanese patent application JP 01 016721 for its part describes the use of gomisin N extracted from Schisandra fruit in order to prevent the effects of aging and arteriosclerosis.

Finally, gomisin A and analogs thereof, essentially extracted from Schisandra, is widely used in Chinese pharmacopeia for treating hepatic disorders, are already used in therapeutics and numerous data regarding their harmlessness are already known and available. Furthermore, the industrialization of gomisin A and of its derivatives is already operational and inexpensive.

There remains a need for novel compounds that make it possible to induce, restore or stimulate the pigmentation of the human skin, body hair and head hair with activity that is more effective than the known compounds, and which has a reinforced action so as to be able to be used in a small amount without any side effects on the skin.

Completely surprisingly, the applicants have demonstrated that certain 2,2′-cyclolignans, such as for example gomisin A and its analogs, exhibit a good pro-pigmenting activity of the skin, body hair or head hair, even at low concentration, without showing cytotoxicity at the active doses.

In particular, the applicants have discovered that gomisin A has the advantage of acting on several major components of the pigmentation mechanism by stimulating:
1) the biosynthesis of melanin by the melanocytes;
2) the formation of a dense dendritic network in the melanocyte;
3) the phagocytic activity of the keratinocytes, thus increasing both the amount of melanin produced and the efficiency of the transfer of melanosomes to the neighboring keratinocytes.

Furthermore, regarding the hair, it has been demonstrated that gomisin A substantially stimulates the pigmentation of the follicle (hair bulb), limits degeneration and increases the survival of the follicle.

One subject of the present invention is therefore the cosmetic use of at least one 2,2′-cyclolignan, or of a cosmetically acceptable salt of said 2,2′-cyclolignan, for inducing, stimulating or restoring the pigmentation of the skin, body hair or head hair.

Within the context of the present invention:
a 2,2′-cyclolignan denotes either the levorotatory form or the dextrorotatory form or a mixture of these two forms of said 2,2′-cyclolignan, when these exist;
an alkyl group denotes a linear or branched, monovalent, saturated, hydrocarbon-based chain comprising from 1 to 6 carbon atoms, the representative elements of which are, for example, the following: methyl, ethyl, n-propyl, isopropyl, n-butyl, sec-butyl, isobutyl, tert-butyl, pentyl or hexyl groups;
the term “alkyl” as defined above retains the same definition when it integrates the name of a group, for example in the alkyloxy group. Thus, among the alkyloxy groups, representative elements are the following: methoxy, ethoxy, n-propoxy, isopropoxy, n-butoxy, isobutoxy, sec-butoxy, tert-butoxy or pentyloxy groups;
gomisin denotes a 2,2′-cyclolignan or a mixture of 2,2′-cyclolignans chosen from gomisin A, gomisin B, gomisin C, gomisin D, gomisin E, gomisin F, gomisin G, gomisin H, gomisin J, gomisin L1, gomisin L2, gomisin O, gomisin T, gomisin R, gomisin S or isomers thereof.

Preferably, the 2,2′-cyclolignan used within the context of the present invention is represented by the formula (I):

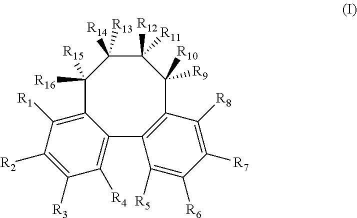

in which R₁, R₂, R₃, R₄, R₅, R₆, R₇, R₈, R₉, R₁₀, R₁₁, R₁₂, R₁₃, R₁₄, R₁₅ and R₁₆ are chosen, independently from one another, as being:
either a hydrogen atom;
or a halogen atom;
or a group chosen from nitro, (C₁-C₆)alkyl, (C₁-C₆)alkyl-COOH, (C₁-C₆)alkyl-COONa, trifluoro(C₁-C₆)alkyl, (C₃-C₆)cycloalkyl, acyl, (C₂-C₆)alkenyl, (C₂-C₆)alkynyl, (C₆-C₁₈)aryl, (C₆-C₁₈)aryl-COOH, (C₆-C₁₈)aryl-COONa, (C₆-C₁₈)aryl-(C₁-C₄)alkyl, (C₁-C₆)alkyl-(C₆-C₁₈)aryl, (C₅-C₁₈)heteroaryl comprising from 1 to 3 heteroatoms, CH(OH)—(C₆-C₁₈)aryl, CO(C₆-C₁₈)-aryl, (CH₂)ₙCONH—(CH₂)ₘ—(C₆-C₁₈)aryl, (CH₂)ₙSO₂—NH—(CH₂)ₘ—(C₆-C₁₈)aryl or (CH₂)ₙCONH—CH(COON)—(CH₂)ₚ—(C₆-C₁₈)aryl groups and where n is between 1 and 4, m is between 0 and 3 and p is between 0 and 2;
or an ORₓ, SRₓ or NRₓR_{y} group in which (i) Rₓ and R_{y}, chosen independently, represent a group chosen from a hydrogen atom, (C₁-C₆)alkyl, (C₃-C₆)cycloalkyl, (C₆-C₁₈)aryl, (C₆-C₁₈)aryl-(C₁-C₄)alkyl, (C₁-C₁₂)alkyl-(C₆-C₁₈)aryl, (C₃-C₆)cycloalkyl-(C₆-C₁₂)aryl, (C₅-C₁₂)heteroaryl comprising from 1 to 3 heteroatoms, NR′R″ or NR′COR″ groups and where R′ and R″, chosen independently, represent a group chosen from a hydrogen atom and (C₁-C₆)alkyl, (C₃-C₆)cycloalkyl and (C₆-C₁₂)aryl groups and aromatic or non-aromatic (C₅-C₁₂) heterocycles comprising 1 to 3 heteroatoms or (ii) Rₓ and R_{y} together form a (C₂-C₆)alkyl or a (C₂-C₆)alkenyl or a (C₂-C₆)heteroalkyl or a (C₂-C₆)heteroalkenyl.

Among the cosmetically acceptable salts, mention will be made, by way of example, of sodium salts, penta-acetates and tribromides. Glycosylated derivatives or esters of said 2,2′-cyclolignan may also be used. Mention will be made, for example, of the esters formed from hemisuccinic acid.

Preferably, the 2,2′-cyclolignan used within the context of the present invention is represented by the formula (II):

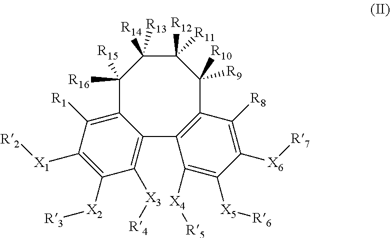

in which
X₁, X₂, X₃, X₄, X₅ and X₆ are chosen, independently from one another, as representing:
   an O or S atom;
   or an NRz group, where Rz is a group chosen from a hydrogen atom and (C₁-C₆)alkyl, (C₃-C₆)cycloalkyl and (C₆-C₁₂)aryl groups and aromatic or non-aromatic (C₅-C₁₂)heterocycles comprising 1 to 3 heteroatoms;
   R₈, R₉, R₁₀, R₁₁, R₁₂, R₁₃, R₁₄, R₁₅ and R₁₆ are chosen, independently from one another, as representing:
   either a hydrogen atom;
   or a halogen atom;
   or a group chosen from nitro, (C₁-C₆)alkyl, (C₁-C₆)alkyl-COOH, (C₁-C₆)alkyl-COONa, trifluoro(C₁-C₆)alkyl, (C₃-C₆)cycloalkyl, acyl, (C₂-C₆)alkenyl, (C₂-C₆)alkynyl, (C₆-C₁₈)aryl, (C₆-C₁₈)aryl-COOH, (C₆-C₁₈)aryl-COONa, (C₆-C₁₈)aryl-(C₁-C₄)alkyl, (C₁-C₆)alkyl-(C₆-C₁₈)aryl, (C₅-C₁₈)heteroaryl comprising from 1 to 3 heteroatoms, CH(OH)—(C₆-C₁₈)aryl, CO(C₆-C₁₈)-aryl, (CH₂)ₙCONH—(CH₂)ₘ—(C₆-C₁₈)aryl, (CH₂)ₙSO₂—NH—(CH₂)ₘ-(C₆-C₁₈)aryl or (CH₂)ₙCONH—CH(COOH)—(CH₂)ₚ—(C₆-C₁₈)aryl groups and where n is between 1 and 4, m is between 0 and 3 and p is between 0 and 2;
R′₁, R′₂, R′₃, R′₄, R′₅, R′₆ and R′₇ are chosen, independently from one another, as:
   representing a group chosen from a hydrogen atom, (C₁-C₆)alkyl, (C₃-C₆)cycloalkyl, (C₆-C₁₈)aryl, (C₆-C₁₈)aryl-(C₁-C₄)alkyl, (C₁-C₁₂)alkyl-(C₆-C₁₈)aryl, (C₃-C₆)cycloalkyl-(C₆-C₁₂)aryl or (C₅-C₁₂)heteroaryl comprising from 1 to 3 heteroatoms groups; or
   forming together or with R₉, R₁₀, R₁₅ or R₁₆ a (C₂-C₆)alkyl or a (C₂-C₆)alkenyl or a (C₂-C₆)heteroalkyl or (C₂-C₆)heteroalkenyl.

More particularly, one subject of the present invention is the cosmetic use, for inducing, stimulating or restoring the pigmentation of the skin, body hair or head hair, of at least one 2,2′-cyclolignan represented by the formula (II) in which X₁, X₂, X₃, X₄, X₅ and X₆ represent an oxygen atom.

Very preferably, one subject of the present invention is the cosmetic use, for inducing, stimulating or restoring the pigmentation of the skin, body hair or head hair, of at least one gomisin, preferably gomisin A.

The 2,2′-cyclolignan according to the present invention and represented by the formula (I) or the formula (II) may be of natural, semi-synthetic or synthetic origin.

The 2,2′-cyclolignan according to the present invention and represented by the formula (I) or the formula (II) may be used pure or as a mixture. A plant extract containing at least one 2,2′-cyclolignan according to the formula (I) or the formula (II) may be used.

Preferably, said plant extract is obtained from the Schisandraceae family. More preferably, said 2,2′-cyclolignan according to the formula (I) or the formula (II) may be obtained from a Schisandra chinensis extract. Thus, a Schisandra chinensis extract may be used for inducing, stimulating or restoring the pigmentation of the skin, body hair or head hair.

The expression “Schisandra extract” is understood to mean an extract of cells of Schisandra and more specifically an extract of cells of at least one plant of the Schisandra genus from the Schisandraceae family. This cell material may be obtained by in vitro or in vivo culturing. The expression “in vitro culturing” is understood to mean all the techniques known to a person skilled in the art which make it possible to artificially obtain a plant or part of a plant. The expression “in vivo culturing” is understood to mean all the culturing techniques which make it possible to obtain a plant or part of a plant. Thus, said extract may be an extract of an organ (for example root, stem, leaf, bark, fruit, seed), of organ cells or of undifferentiated cells of at least one plant of the Schisandra genus from the Schisandraceae family. This extract is enriched in 2,2′-cyclolignan according to the formula (I) or the formula (II) in variable proportions depending on the type of extract.

The Schisandra extract according to the present invention may be used in crude or purified form. The purification of a Schisandra extract according to the present invention makes it possible to avoid problems of toxicity of said crude extract. The purification of a Schisandra extract according to the present invention may thus consist:
either in concentrating a 2,2′-cyclolignan or a mixture of 2,2′-cyclolignans according to the formula (I) or the formula (II) present in said extract;
or in obtaining a pure gomisin.

In order to carry out the purification of a Schisandra extract, any method of extraction or of purification known to a person skilled in the art may be used. In particular, according to the present invention, the Schisandra extract may be obtained by alcoholic (especially methanolic or ethanolic) or aqueous extraction or by extraction using solvents such as ketones, esters, ethers, polyols, chlorinated solvents and mixtures of at least two of these solvents, such as aqueous/alcoholic extraction.

Thus, another subject of the present invention is the use of at least one 2,2′-cyclolignan as defined above, or of a pharmaceutically acceptable salt, for the preparation of a medicament intended for the preventative or curative treatment of any disease that induces a depigmentation of the skin, body hair or head hair.

Examples of pharmaceutically acceptable salts are presented in the publication by Berge et al., “Pharmaceutically acceptable salts”, J. Pharm. Sci. 1997, 66, 1-19.

By way of example of diseases that induce a depigmentation of the skin, body hair or head hair which may be treated according to the present invention, mention may be made of the following diseases: vitiligo, albinism, atopic dermatitis, psoriasis, leprosy, kwashiorkor, phenylketonuria, tuberous sclerosis, piebaldism, Waardenburg syndrome and Pallister-Killian syndrome.

Another subject of the present invention is the use of at least one 2,2′-cyclolignan as defined above, or of a pharmaceutically acceptable salt, for the preparation of a dermatological composition intended for the preventative or curative treatment of any disease that induces a depigmentation of the skin, body hair or head hair.

Examples of pharmaceutically acceptable salts are presented in the publication by Berge et al., “Pharmaceutically acceptable salts”, J. Pharm. Sci. 1997, 66, 1-19.

By way of example of diseases that induce a depigmentation of the skin, body hair or head hair which may be treated according to the present invention, mention may be made of the following diseases: vitiligo, albinism, atopic dermatitis, psoriasis, leprosy, kwashiorkor, phenylketonuria, tuberous sclerosis, piebaldism, Waardenburg syndrome and Pallister-Killian syndrome.

The 2,2′-cyclolignan described in the context of the present invention may therefore be used in a cosmetic, pharmaceutical or dermatological composition. The amount of 2,2′-cyclolignan that can be used in such compositions is of course dependent on the desired effect and may therefore vary to a wide extent. Preferably, it is possible to use a 2,2′-cyclolignan according to the invention, or a plant extract containing it, in an amount representing from 0.01 to 5% of the total weight of the cosmetic or pharmaceutical composition prepared, preferably in an amount representing from 0.01% to 2.5% of the total weight of the composition and, more preferably, in an amount representing from 0.01% to 0.25% of the total weight of the composition.

Advantageously, the 2,2′-cyclolignan according to the present invention may be associated, in the cosmetic or pharmaceutical composition, with a vehicle that is compatible with and suitable for the chosen method of administration. Preferably, said cosmetic or pharmaceutical composition is adapted for a topical application.

For a use according to the invention, a composition may be in the form of creams, gels, lotions, milks, oil-in-water or water-in-oil emulsions, solutions, ointments, sprays, body oils, hair lotions, shampoos, after-shave lotions, soaps, lip protection sticks and makeup sticks and pencils.

In gel form, a composition according to the invention comprises suitable excipients such as cellulose esters or other gelling agents, such as carbopol or guar gum.

A composition according to the invention may also be in the form of a lotion or solution in which an extract and/or at least one 2,2′-cyclolignan according to the invention is in encapsulated form, for example in microspheres. These microspheres may be constituted, for example, of fatty substances, agar and water. A pigmentation-promoting agent according to the invention may also be incorporated into vectors such as liposomes, glycospheres, cyclodextrins, into chylomicrons, macro-, micro- or nanoparticles and also macro-, micro- and nanocapsules, and may also be adsorbed onto pulverulent organic polymers, talcs, bentonites and other mineral supports. These emulsions show good stability and may be kept for the time required for use at temperatures of between 0 and 50° C. without any sedimentation of the constituents or phase separation taking place.

A composition according to the present invention may also contain additives or adjuvants that are common in cosmetology, for instance antibacterial agents or fragrances, but also extracted and/or synthetic lipids, gelling and viscosity-increasing polymers, surfactants and emulsifiers, water-soluble or liposoluble active principles, plant extracts, tissue extracts, marine extracts or synthetic active agents.

A composition according to the present invention may also comprise other additional active principles chosen for their action, for example for antisun protection, the anti-wrinkle effect, the free-radical-scavenging and antioxidant activity, the anti-irritant activity, cell nutrition, cell respiration, cell hydration and regeneration, anti-seborrheic treatments, and also other active principles with action on skin tonicity or hair protection.

A composition according to the present invention is preferably to be used daily by applying it one or more times a day.

A composition according to the present invention is very well tolerated, shows no phototoxicity and its application to the skin, for prolonged periods of time, involves no systemic effect.

The examples that follow illustrate the invention in a non-limiting manner.

### DESCRIPTION OF THE FIGURE

FIG. 1 illustrates the evaluation of the melanin produced in normal human melanocytes in the presence of gomisin A.

### EXAMPLE 1

### Demonstration of the Activity on Melanogenesis in Melanocyte Cultures

A biological test demonstrated the stimulatory activity of gomisin A on melanin synthesis.

The melanogenesis-stimulating effect of gomisin A was measured on B16 melanocyte cultures.

For gomisin A, the following were determined:
after culturing for 10 days under standard conditions, in 24-well plates, in Promocell medium free of “phorbol myristate acetate” (PMA):

the cytotoxicity, by estimating the reduction of “methyl thiazolyl tetrazolium” (MTT);

the amount of proteins, by assay according to the Bradford method and observation of the cell layer;

the amount of melanin present in the cultures, by spectrophotometric measurement of the melanin produced, after alkaline extraction, relative to 100% of the control (the control corresponds to the test performed without test compound).

The results are collated in the tables 1 and 2 below:

TABLE 1
Effects of gomisin A on the viability of B16 melanocytes during culturing (72 h contact).
MTT test and morphological observations
Gomisin A (2 mg/ml stock solution in ethanol)

| mg/ml | 0 | 9.1E−06 | 3E−05 | 8E−05 | 2E−04 | 7E−04 | 0.002 | 0.007 | 0.02 | 0 |
|---|---|---|---|---|---|---|---|---|---|---|
| | 1.864 | 1.951 | 1.764 | 1.899 | 1.721 | 1.844 | 1.920 | 2.046 | 1.988 | 1.760 |
| | 1.840 | 1.879 | 1.910 | 1.829 | 1.807 | 1.896 | 1.933 | 2.061 | 2.085 | 1.926 |
| | 1.795 | 1.901 | 1.740 | 1.770 | 1.787 | 1.987 | 1.942 | 1.966 | 2.182 | 1.836 |
| | 1.895 | 1.837 | 1.961 | 1.964 | 1.878 | 1.976 | 2.022 | 2.046 | 2.227 | 1.964 |
| | 1.966 | 1.963 | 1.784 | 1.891 | 1.932 | 1.996 | 2.112 | 2.137 | 2.046 | 2.080 |
| | 1.923 | 1.930 | 1.853 | 1.965 | 1.972 | 2.147 | 2.077 | 2.227 | 2.241 | 2.016 |
| average | 1.905 | 1.910 | 1.835 | 1.886 | 1.850 | 1.974 | 2.001 | 2.081 | 2.128 | |
| viability (%) | 100 | 100 | 96 | 99 | 97 | 104 | 105 | 109 | 112 | |
| Observations | + | + | + | + | + | + | + | + | + | |

TABLE 2
Effects of gomisin A on the synthesis of melanin by B16 melanocytes
Melanin assay - plate 1

| | | | | | | | | p | | |
|---|---|---|---|---|---|---|---|---|---|---|
| | | | Melanin | | | % | | without | Proteins | |
| Treatment | Conc. | | (μg/ml) | sd | n | Control | p | IBMX | (mg/ml) | MTT (%) |
| Control | — | | 13.79 | 1.72 | 3 | 100 | — | — | 1.170 | 100 |
| IBMX | 200 | μM | 173.77 | 11.61 | 3 | 1261 | <0.01 | — | 0.917 | 99 |
| Gomisin A | 0.02 | mg/ml | 27.73 | 0.76 | 3 | 201 | <0.01 | <0.01 | 0.992 | 102 |
| | 0.0067 | mg/ml | 22.66 | 2.72 | 3 | 164 | >0.05 | <0.01 | 1.000 | 99 |
| | 0.0022 | mg/ml | 16.62 | 0.17 | 3 | 121 | >0.05 | >0.05 | 1.003 | 102 |

Gomisin A therefore caused a significant increase in melanin production in the melanocyte cultures treated at non-toxic doses. The product did not moreover show an effect on the proliferation/protein synthesis of keratinocytes.

### EXAMPLE 2

### Demonstration of the Activity on Melanogenesis in Melanocyte Cultures

A biological test demonstrated the stimulatory activity of gomisin C on melanin synthesis. The melanogenesis-stimulating effect of gomisin C was measured on B16 melanocyte cultures.

For gomisin C, the following were determined:
after culturing for 10 days under standard conditions, in 24-well plates, in Promocell medium free of “phorbol myristate acetate” (PMA):

the cytotoxicity, by estimating the reduction of “methyl thiazolyl tetrazolium” (MTT), the amount of proteins, by assay according to the Bradford method and observation of the cell layer;

the amount of melanin present in the cultures, by spectrophotometric measurement of the melanin produced, after alkaline extraction, relative to 100% of the control (the control corresponds to the test performed without test compound).

The results are collated in the tables 3 and 4 below:

TABLE 3
Effects of gomisin C on the viability of B16 melanocytes during culturing (72 h contact).
MTT test and morphological observations
Gomisin C

| % | 0 | 2.56E−07 | 1E−06 | 6E−06 | 3E−05 | 0.0002 | 0.0008 | 0.004 | 0.02 | 0 |
|---|---|---|---|---|---|---|---|---|---|---|
| | 2.753 | 2.786 | 2.793 | 2.778 | 2.789 | 2.786 | 2.834 | 1.871 | 1.208 | 2.699 |
| | 2.791 | 2.781 | 2.813 | 2.796 | 2.795 | 2.783 | 2.824 | 1.873 | 1.146 | 2.738 |
| | 2.798 | 2.804 | 2.827 | 2.801 | 2.818 | 2.780 | 2.873 | 1.954 | 1.205 | 2.779 |
| average | 2.760 | 2.790 | 2.811 | 2.792 | 2.801 | 2.783 | 2.844 | 1.899 | 1.136 | |
| viability (%) | 100 | 101 | 102 | 101 | 101 | 101 | 103 | 69 | 43 | |
| Observations | + | + | + | + | + | + | + | − | − | |

TABLE 4
Effects of gomisin C on the synthesis of melanin by B16 melanocytes
Melanin assay

| | | | | | | | | Total | |
|---|---|---|---|---|---|---|---|---|---|
| | | | Melanin | | | | | proteins | % cell |
| Treatment | Conc. | | (μg/ml) | sd | n | % Control | p | (mg/ml) | viability |
| Control | — | | 16.12 | 0.58 | 3 | 100 | — | 1.297 | |
| DMSO (vehicle) | 0.016% | | 16.04 | 0.72 | 3 | 100 | p > 0.05 | 1.374 | 101 |
| IBMX | 200 | μM | 90.45 | 0.85 | 3 | 561 | p < 0.01 | 1.428 | 99 |
| | 40 | μM | 25.91 | 1.62 | 3 | 161 | p < 0.01 | 1.338 | 94 |
| | 8 | μM | 18.88 | 0.74 | 3 | 117 | p < 0.01 | 1.292 | 96 |
| Gomisin C | 8 | μg/ml | 19.44 | 0.42 | 3 | 121 | p < 0.01 | 1.299 | 101 |
| | 2 | μg/ml | 17.46 | 0.93 | 3 | 108 | p > 0.05 | 1.294 | 97 |
| | 0.4 | μg/ml | 16.66 | 0.32 | 3 | 103 | p > 0.05 | 1.220 | 102 |

Gomisin C therefore caused a significant increase in melanin production in the melanocyte cultures treated at non-toxic doses. The product did not moreover show an effect on the proliferation/protein synthesis of keratinocytes.

### EXAMPLE 3

### Evaluation of the Melanin Produced in Normal Human Melanocytes in the Presence of Gomisin A

The product GPN000715 corresponds to gomisin A.

Treatment of 2D Melanocytes by the Test and Reference Formulations:

The study was carried out on Caucasian primary melanocytes during culturing. The test and reference formulations were applied in the presence of L-dopa which is the substrate of tyrosinase (50 μM) (Sigma, D9628-5G) for 4 days, the culture medium having been renewed after 2 days. The product GPN000715 was applied at three different concentrations: 10 μM, 30 μM and 80 μM. An application of 100 nM of the reference element (MSH) was carried out, and also an application of 0.1 mM of arbutin. The untreated control cells (CTL) stayed in the culture medium.

Quantification of Melanin:

The melanocytes were seeded in 6-well plates at a cell density of +/−100 000 cells. One week after seeding the cells, the test and reference products were applied, in the presence of L-dopa. After 4 days of treatment, cell lysis was carried out using a solution of NaOH (1N) (Merck, 109956) over 24 hours, at 37° C. The cell lysates were then centrifuged for 10 minutes, then 120 μl of each lysate was transferred into a 96-well plate in order to read the absorbance at 450 nm. The melanin content of each sample was quantified by comparison with the absorbances from the standard curve made with synthetic melanin (Sigma, M8631-100MG), ranging from 0 to 100 μg/ml.

Results

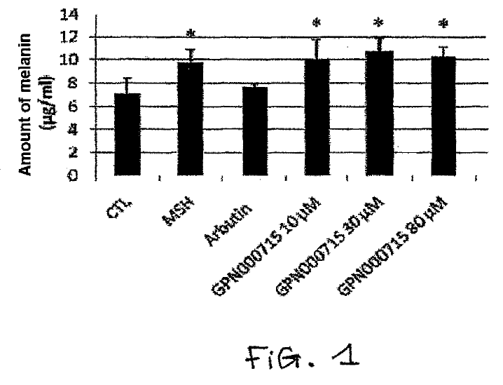

FIG. 1 represents a quantification of the production of melanin by melanocytes, in a single layer, after 4 days of application in the presence of the test and reference elements. Each bar represents the standard error (n=3). The Fischer comparison test was used for the statistical analysis of the data (the asterisk “*” signifies p<0.01 vs control).

Thus, it is observed that at a concentration of 30 μM, gomisin A significantly increases the amount of melanin in normal human melanocytes, by the order of 40%.

1. The cosmetic use of at least one 2,2′-cyclolignan, or of a cosmetically acceptable salt of said 2,2′-cyclolignan, for inducing, stimulating or restoring the pigmentation of the skin, body hair or head hair.

2. The use as claimed in claim 1, characterized in that the 2,2′-cyclolignan is represented by the formula (1): 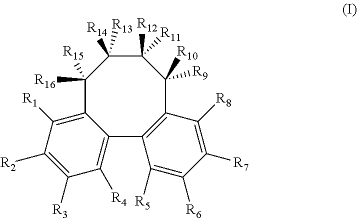
in which R₁, R₂, R₃, R₄, R₅, R₆, R₇, R₈, R₉, R₁₀, R₁₁, R₁₂, R₁₃, R₁₄, R₁₅ and R₁₆ are chosen, independently from one another, as being:
either a hydrogen atom;
or a halogen atom;
or a group chosen from nitro, (C₁-C₆)alkyl, (C₁-C₆)alkyl-COOH, (C₁-C₆)alkyl-COONa, trifluoro(C₁-C₆)alkyl, (C₃-C₆)cycloalkyl, acyl, (C₂-C₆)alkenyl, (C₂-C₆)alkynyl, (C₆-C₁₈)aryl, (C₆-C₁₈)aryl-COOH, (C₆-C₁₈)aryl-COONa, (C₆-C₁₈)aryl-(C₁-C₄)alkyl, (C₁-C₆)alkyl-(C₆-C₁₈)aryl, (C₅-C₁₈)heteroaryl comprising from 1 to 3 heteroatoms, CH(OH)—(C₆-C₁₈)aryl, CO(C₆-C₁₈)-aryl, (CH₂)ₙCONH—(CH₂)ₘ—(C₆-C₁₈)aryl, (CH₂)ₙSO₂—NH—(CH₂)ₘ—(C₆-C₁₈)aryl or (CH₂)ₙCONH—CH(COOH)—(CH₂)ₚ—(C₆-C₁₈)aryl groups and where n is between 1 and 4, m is between 0 and 3 and p is between 0 and 2;
or an ORₓ, SRₓ or NRₓR_{y} group in which (i) Rₓ and R_{y}, chosen independently, represent a group chosen from a hydrogen atom, (C₁-C₆)alkyl, (C₃-C₆)cycloalkyl, (C₆-C₁₈)aryl, (C₆-C₁₈)aryl-(C₁-C₄)alkyl, (C₁-C₁₂)alkyl-(C₆-C₁₈)aryl, (C₃-C₆)cycloalkyl-(C₆-C₁₂)aryl, (C₅-C₁₂)heteroaryl comprising from 1 to 3 heteroatoms, NR′R″ or NR′COR″ groups and where R′ and R″, chosen independently, represent a group chosen from a hydrogen atom and (C₁-C₆)alkyl, (C₃-C₆)cycloalkyl and (C₆-C₁₂)aryl groups and aromatic or non-aromatic (C₅-C₁₂)heterocycles comprising 1 to 3 heteroatoms or (ii) Rₓ and R_{y} together form a (C₂-C₆)alkyl or a (C₂-C₆)alkenyl or a (C₂-C₆)heteroalkyl or a (C₂-C₆)heteroalkenyl.

3. The use as claimed in claim 1, characterized in that the 2,2′-cyclolignan is represented by the formula (II): 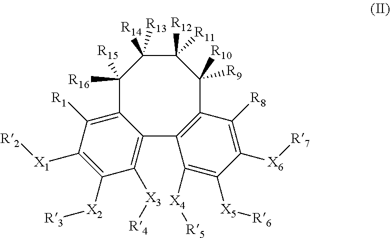
in which
X₁, X₂, X₃, X₄, X₅ and X₆ are chosen, independently from one another, as representing:
an O or S atom;
or an NRz group, where Rz is a group chosen from a hydrogen atom and (C₁-C₆)alkyl, (C₃-C₆)cycloalkyl and (C₆-C₁₂)aryl groups and aromatic or non-aromatic (C₅-C₁₂)heterocycles comprising 1 to 3 heteroatoms;
R_{8,} R₉, R₁₀, R₁₁, R₁₂, R₁₃, R₁₄, R₁₅ and R₁₆ are chosen, independently from one another, as representing:
either a hydrogen atom;
or a halogen atom;
or a group chosen from nitro, (C₁-C₆)alkyl, (C₁-C₆)alkyl-COOH, (C₁-C₆)alkyl-COONa, trifluoro(C₁-C₆)alkyl, (C₃-C₆)cycloalkyl, acyl, (C₂-C₆)alkenyl, (C₂-C₆)alkynyl, (C₆-C₁₈)aryl, (C₆-C₁₈)aryl-COOH, (C₆-C₁₈)aryl-COONa, (C₆-C₁₈)aryl-(C₁-C₄)alkyl, (C₁-C₆)alkyl-(C₆-C₁₈)aryl, (C₅-C₁₈)heteroaryl comprising from 1 to 3 heteroatoms, CH(OH)—(C₆-C₁₈)aryl, CO(C₆-C₁₈)-aryl, (CH₂)ₙCONH—(CH₂)ₘ—(C₆-C₁₈)aryl, (CH₂)ₙSO₂—NH—(CH₂)ₘ—(C₆-C₁₈)aryl or (CH₂)ₙCONH—CH(COOH)—(CH₂)ₚ—(C₆-C₁₈)aryl groups and where n is between 1 and 4, m is between 0 and 3 and p is between 0 and 2;
R′₁, R′₂, R′₃, R′₄, R′₅, R′₆ and R′₇ are chosen, independently from one another as:
representing a group chosen from a hydrogen atom, (C₁-C₆)alkyl, (C₃-C₆)cycloalkyl, (C₆-C₁₈)aryl, (C₆-C₁₈)aryl-(C₁-C₄)alkyl, (C₁-C₁₂)alkyl-(C₆-C₁₈)aryl, (C₃-C₆)cycloalkyl-(C₆-C₁₂)aryl or (C₅-C₁₂)heteroaryl comprising from 1 to 3 heteroatoms groups; or
forming together or with R₉, R₁₀, R₁₅ or R₁₆ a (C₂-C₆)alkyl or a (C₂-C₆)alkenyl or a (C₂-C₆)heteroalkyl or (C₂-C₆)heteroalkenyl.

4. The use as claimed in claim 3, characterized in that X₁, X₂, X₃, X₄, X₅ and X₆ represent an oxygen atom.

5. The use as claimed in claim 1, characterized in that the 2,2′-cyclolignan is a gomisin.

6. The use as claimed in claim 5, characterized in that the gomisin is gomisin A.

7. The use as claimed in claim 1, characterized in that the 2,2′-cyclolignan(s) is (are) present in a cosmetic composition in an amount representing from 0.01 to 5% of the total weight of the composition.

8. The use of at least one 2,2′-cyclolignan, or of a pharmaceutically acceptable salt of said 2,2′-cyclolignan, for the preparation of a medicament intended for the preventative or curative treatment of any disease that induces a depigmentation of the skin, body hair or head hair.

9. The use of at least one 2,2′-cyclolignan, or of a pharmaceutically acceptable salt of said 2,2′-cyclolignan, for the preparation of a medicament intended for treating one of the following diseases: vitiligo, albinism, atopic dermatitis, psoriasis, leprosy, kwashiorkor, phenylketonuria, tuberous sclerosis, piebaldism, Waardenburg syndrome and Pallister-Killian syndrome.

10. The use as claimed in claim 8, characterized in that the 2,2′-cyclolignan(s) is (are) present in an amount representing from 0.01 to 5% of the total weight of the medicament.

11. The use of at least one 2,2′-cyclolignan, or of a pharmaceutically acceptable salt of said 2,2′-cyclolignan, for the preparation of a dermatological composition intended for the preventative or curative treatment of any disease that induces a depigmentation of the skin, body hair or head hair.

12. The use of at least one 2,2′-cyclolignan, or of a pharmaceutically acceptable salt of said 2,2′-cyclolignan, for the preparation of a dermatological composition intended for treating one of the following diseases: vitiligo, albinism, atopic dermatitis, psoriasis, leprosy, kwashiorkor, phenylketonuria, tuberous sclerosis, piebaldism, Waardenburg syndrome and Pallister-Killian syndrome.

13. The use as claimed in claim 11, characterized in that the 2,2′-cyclolignan(s) is (are) present in an amount representing from 0.01 to 5% of the total weight of the composition.

14. The use as claimed in claim 9, characterized in that the 2,2′-cyclolignan(s) is (are) present in an amount representing from 0.01 to 5% of the total weight of the medicament.

15. The use as claimed in claim 12, characterized in that the 2,2′-cyclolignan(s) is (are) present in an amount representing from 0.01 to 5% of the total weight of the composition.
